(19) United States
(12) Patent Application Publication
(10) Pub. No.: US 2024/0180785 A1
(43) Pub. Date: Jun. 6, 2024

(54) MONITORING DOSING REGIMENS OF CONSUMABLES

(71) Applicant: HERO HEALTH INC., New York, NY (US)

(72) Inventors: Nemanja JOKSOVIC, New York, NY (US); Michal LEVIN, Sunnyvale, CA (US); Kevin M. FARINO, Beverly Hills, CA (US); Kalyan C. VEPURI, New York, NY (US)

(21) Appl. No.: 18/444,084

(22) Filed: Feb. 16, 2024

Related U.S. Application Data
(63) Continuation of application No. PCT/US2022/040575, filed on Aug. 17, 2022.
(60) Provisional application No. 63/234,101, filed on Aug. 17, 2021.
(60) Provisional application No. 63/357,367, filed on Jun. 30, 2022.

(51) Int. Cl. A61J 7/04; G16H 20/13

(52) U.S. Cl. CPC A61J 7/049; A61J 7/0436; G16H 20/13; A61J 2200/30; A61J 2200/70; A61J 2200/74

(57) ABSTRACT

According to one aspect, a method of monitoring a dosing regimen of consumables may include actuating a retrieval mechanism to direct at least a portion of a scheduled dose of consumable media, according to a predetermined schedule, from one or more containers to a dispensing zone, receiving a first signal indicative of a quantity of the consumable media directed from the one or more containers to the dispensing zone, receiving a second signal indicative of a quantity of the scheduled dose of the consumable media consumed by a user associated with the scheduled dose, and based on the quantity of the consumable media directed to the dispensing zone and the quantity of the consumable media consumed by the user, determining a degree of compliance for the user with respect to the scheduled dose.

### CROSS-REFERENCE TO RELATED APPLICATIONS

This application is a continuation of International Patent Application No. PCT/US2022/040575, filed Aug. 17, 2022, which claims the benefit of priority to U.S. Provisional Application Ser. No. 63/234,101, filed Aug. 17, 2021, and U.S. Provisional Application Ser. No. 63/357,367, filed Jun. 30, 2022, with the entire contents of each of these applications hereby incorporated herein by reference.

### BACKGROUND

Automated pill dispensing is useful for improving the likelihood of user compliance with respect to proper timing and dosage of medications. The potential for improved compliance is manifest, for example, in medication regimens that involve several different types of medications, scheduled to be taken at various times throughout the day or night. While techniques accurately and automatically retrieving pills for dispensing can make it easier to manage complex medication regimens, automatic pill dispensing can be time-consuming and may require inconvenient intervention by the user or third parties. These issues can frustrate some users to the point of decreased reliance on automated pill dispensing to manage medication regimens, potentially decreasing user-compliance with the medication regimens. Thus, there remains a need for improved techniques for monitoring user compliance with dosing regimens of consumables managed by automatic pill retrieval and dispensing.

### SUMMARY

According to one aspect, a method of monitoring a dosing regimen of consumables may include actuating a retrieval mechanism to direct at least a portion of a scheduled dose of consumable media, according to a predetermined schedule, from one or more containers to a dispensing zone, receiving a first signal indicative of a quantity of the consumable media directed from the one or more containers to the dispensing zone, receiving a second signal indicative of a quantity of the scheduled dose of the consumable media consumed by a user associated with the scheduled dose, and based on the quantity of the consumable media directed to the dispensing zone and the quantity of the consumable media consumed by the user, determining a degree of compliance for the user with respect to the scheduled dose.

In certain implementations, the degree of compliance may include non-compliance, compliance, and one or more degrees of partial compliance with respect to the scheduled dose.

In some implementations, the first signal may include feedback from an optical sensor, a weight sensor, or a combination thereof.

In certain implementations, the second signal corresponds to removal of the consumable media from the dispensing zone.

In some implementations, the second signal includes feedback from an optical sensor, a weight sensor, or a combination thereof. For example, the second signal may include feedback from a sensor detecting removal of a cup from the dispensing zone.

In certain implementations, the scheduled dose of consumable media may include one or more consumables stored apart from the one or more containers and separately accessible by the user.

In some implementations, the method may further include initiating a remedial action when the degree of compliance is incomplete for the user with respect to the scheduled dose. For example, the remedial action may include sending one or more alerts to a user interface associated with the user. Further, or instead, the remedial action may include sending one or more prompts to a user interface associated with the user. Additionally, or alternatively, the remedial action may include sending the one or more prompts to an application on a remote device associated with the user. In some instances, sending the one or more prompts to the user interface may include sending a first set of prompts if the first signal indicates that at least one of the one or more containers is empty and otherwise sending a second set of prompts, different from the first set of prompts, if the first signal indicates that the quantity of the consumable media directed to the dispensing zone is less than the scheduled dose of the consumable media. Further, or instead, the second signal may include one or more user inputs at the user interface in response to the one or more prompts. Still further or instead, the one or more user inputs in the second signal may correspond to one or more of a type or a number of the consumable media consumed by the user at a scheduled time associated with the scheduled dose of the consumable media. In certain instances, the one or more user inputs in the second signal may include an indication that at least a portion of the consumable media is no longer needed. Additionally, or alternatively, the one or more user inputs in the second signal may include an instruction to exclude at least one of the one or more containers from subsequent attempts to direct the consumable media to the dispensing zone.

In certain implementations, the method may further include updating an adherence metric according to the degree of compliance following the remedial action, the adherence metric associated with the dosing regimen of consumables associated with the user.

According to another aspect, a computer program product encoded on one or more non-transitory computer storage media may include instructions that, when executed by one or more computing devices, cause the one or more computing devices to perform operations including receiving a first signal indicative of a quantity of consumable media directed from one or more containers to a dispensing zone, receiving a second signal indicative of a quantity of the consumable media consumed by a user associated with a scheduled dose of the consumable media, and based on the quantity of the consumable media directed from the one or more containers to the dispensing zone and the quantity of the consumable media consumed by the user, determining a degree of compliance for the user with respect to the scheduled dose.

According to yet another aspect, a dispensing device may include a plurality of containers, a retrieval arm, a positioner coupled to the retrieval arm and actuatable to move the retrieval arm in one or more of the plurality of containers, and a controller in communicating relationship with the positioner, the controller configured to actuate the positioner to direct at least one portion of a scheduled dose of consumable media, according to a predetermined schedule, from one or more of the plurality of containers to a dispensing zone, receive a first signal indicative of a quantity measured by a sensor, receive a second signal indicative of an input from a user interface, and based on the first signal and the second signal, determine a degree of compliance for a user associated with the scheduled dose.

In certain implementations, the degree of compliance may include non-compliance, compliance, and one or more degrees of partial compliance with respect to the scheduled dose.

In some implementations, the second signal may be indicative of a degree of consumption of the scheduled dose.

In some implementations, the second signal may be indicative of a time of consumption of the at least one portion of the scheduled dose of the consumable media.

In certain implementations, the scheduled dose of the consumable media may include one or more consumables stored apart from the plurality of containers and separately accessible by the user, and the second signal is indicative of consumption of the one or more consumables stored apart from the plurality of containers and separately accessible by the user.

In some implementations, the dispensing device may further include the sensor, wherein the sensor includes one or more of an optical sensor or a weight sensor.

In certain implementations, the dispensing device may further include a network interface for coupling the controller in a communicating relationship with the user interface over a data network.

In some implementations, the dispensing device may further include the user interface, wherein the user interface is in electrical communication with the controller.

### BRIEF DESCRIPTION OF THE DRAWINGS

FIG. 1A is front perspective view of a dispensing device of the system of FIG. 1.

FIG. 1B is a rear perspective view of the dispensing device of FIG. 1A, shown with a portion of a housing removed.

FIG. 1C is a schematic representation of a retrieval arm, a carrier, a container, and a controller of the dispensing device of FIG. 1A.

FIG. 1D is a top view of the carrier and the container of the dispensing device of FIG. 1A.

FIG. 2 is a schematic representation of a system for monitoring a dosing regimen of consumables.

FIG. 3 is a flow chart of an exemplary method of monitoring a dosing regimen of consumables.

Like reference symbols in the various drawings indicate like elements.

### DETAILED DESCRIPTION

Embodiments will now be described more fully hereinafter with reference to the accompanying figures, in which example embodiments are shown. The foregoing may, however, be embodied in many different forms and should not be construed as limited to the embodiments set forth herein.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and the term “and” should generally be understood to mean “and/or.”

As used herein, the term “user” shall be understood to refer to a person who ultimately consumes (e.g., ingests, applies, etc.) consumable media for his or her own benefit, for therapeutic purposes and often—although not exclusively—at the direction of a healthcare professional. In certain instances, the user may be assisted by one or more healthcare professionals, caregivers, family members, friends, etc. However, for the sake of clear and efficient description, these parties acting on behalf of the user to assist the user in consuming the consumable media automatically dispensed according to the techniques described herein shall be understood to be acting as a proxy for the user and, thus, shall be referred to as the user unless a contrary intent is expressly indicated or made clear from the context.

In the description that follows, the term “consumable media” shall be generally understood to include any one or more of various, different types of media that may be ingested, applied, and/or otherwise used by a user for a therapeutic purpose. Thus, for example, consumable media may often include pills of any form, shape, and type intended to be swallowed, chewed, and/or dissolved in the user's mouth. Additionally, or alternatively, consumable media may include topical treatment that may be applied to the user's skin. Further, or instead, consumable media may include a measured quantity of a liquid to be swallowed by the user. More generally, unless otherwise specified or made clear from the context, consumable media shall be understood to include any one or more of various, different therapeutic media that may be dispensed in doses to a user in furtherance of some form of improvement and/or maintenance of one or more aspects of the user's health. For the sake of clear and efficient description, the terms “consumable media” and “pills” may be used interchangeably herein, as dictated by the context, and shall not be understood to be limiting, unless an intention to the contrary is expressly indicated.

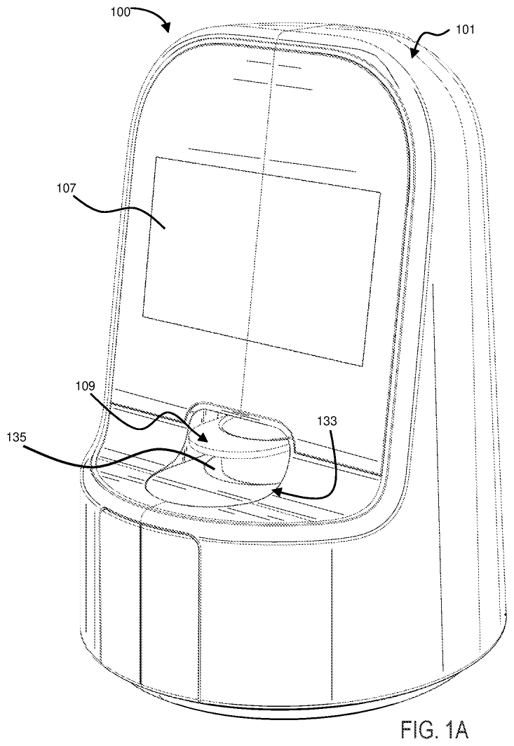

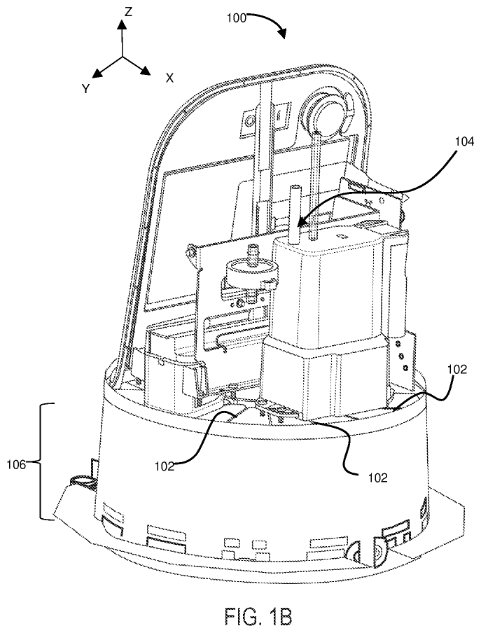

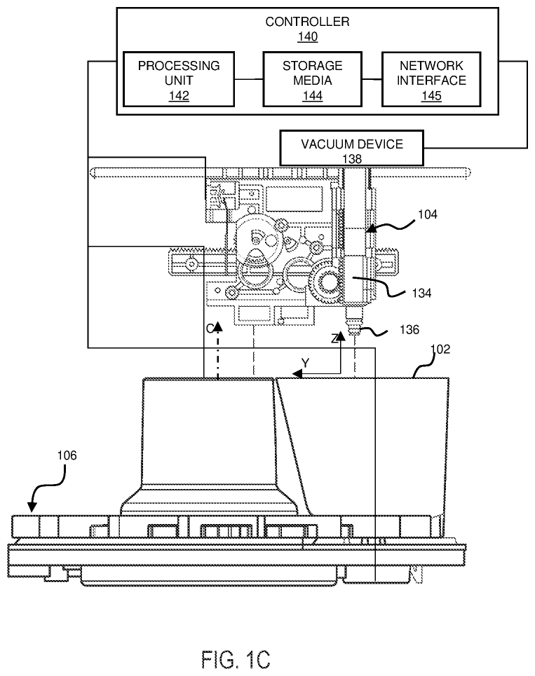

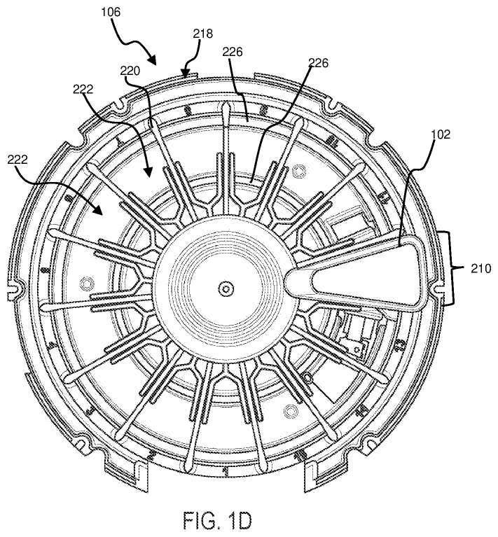

Referring now to FIGS. 1A-1D, a dispensing device 100 for dispensing pills may include a housing 101 disposed about a plurality of containers 102 (with each instance of the plurality of containers 102 referred to as a container 102), a retrieval arm 104, a carrier 106, a user interface 107, and a controller 140, with the housing 101 generally providing restricted access at least to contents of the plurality of containers 102. Pills may be sorted into the plurality of containers 102, with each one of the plurality of containers 102 carrying a different type of pill to be dispensed to a user of the dispensing device 100. The carrier 106 may support the plurality of containers 102, and the carrier 106 may be movable to adjust positions of the plurality of containers 102 relative to a predetermined position 210. In use, as described in greater detail below, movement of the retrieval arm 104 may be coordinated with movement of the carrier 106 to carry out pick-and-place retrieval of individual pills from the plurality of containers 102 and ultimately deliver the retrieved pills to a dispensing zone 133 (e.g., into a dispensing cup 109 positioned at the dispensing zone 133), where an end-user may access the retrieved pills according to a predetermined schedule or in response to an ad hoc request. As described in greater detail below, the controller 140 of the dispensing device may carry out one or more aspects of various, different techniques for determining a user's degree of compliance with respect to a scheduled dose in a dosing regimen and/or for determining a user's overall adherence to the dosing regimen. Tracking these parameters and making these parameters available to the user may be useful for providing the user with feedback regarding the dosing regimen. This feedback, by itself, may provide the user with transparent metrics that may reduce the potential for user frustration with automated dispensing (e.g., time required, operation of the dispensing device 100, convenience relative to the user's other activities, etc.) while encouraging the user to take steps to improve adherence to the dosing regimen. As also described in greater detail below, the controller 140 may further, or instead, initiate one or more remedial actions that may assist the user in achieving improved degrees of compliance with scheduled doses and, ultimately, improved adherence to the dosing schedule. These remedial actions may further reduce the likelihood of user frustration with automated dispensing, as these remedial actions may be generally directed to incorporating user feedback into the dosing regimen, guiding the user with respect to use of the dispensing device 100, and/or making the dispensing device 100 more convenient to use.

In general, the carrier 106 may be rotatable about an axis C, and the plurality of containers 102 may be disposed at different angular positions about the axis C such that rotation of the carrier 106 changes the position of the plurality of containers 102 relative to the predetermined position 210. Stated differently, carrier 106 may be rotated about the axis C to change which instance of the plurality of containers 102 is beneath the retrieval arm 104. While the plurality of containers 102 may be arranged at different angular positions about the axis C (similar to numbers on a clock), it shall be appreciated that only a single instance of the plurality of containers 102 (the container 102 located at the predetermined position 210) is shown in FIGS. 1B, 1C, and 1D to facilitate clear illustration and efficient description

In general, the retrieval arm 104 may be movable into one or more instances of the plurality of containers 102 to retrieve a single pill with each successful retrieval attempt. While the retrieval arm 104 may be movable into the plurality of containers 102 along any angular position relative to the predetermined position 210, the retrieval arm 104 may be advantageously movable into the container 102 at the predetermined position 210. That is, at the predetermined position 210, the retrieval arm 104 may movable into the container 102 along a z-axis parallel to the center axis C of the turntable 220 such that the force exerted by the retrieval arm 104 on the pills in the container 102 may be detected by a weight sensor. Further, in some instances, this detected force may be used to limit movement of the retrieval arm 104 to reduce the likelihood of damaging the retrieval arm 104, the container 102, or pills within the container 102. Continuing with this example, to retrieve pills in each of the other instances of the plurality of containers 102, another instance of the plurality of containers 102 may be moved to the predetermined position 210, where the retrieval arm 104 may again be moved along the z-axis to repeat the process of pill retrieval. It shall be appreciated that such movement of the retrieval arm 104 along the z-axis at the predetermined position 210 may be carried out as many times as necessary and in as many different instances of the plurality of containers 102 as necessary to retrieve the type and number of pills for dispensing to the end-user, such as may be dictated by a predetermined schedule or an ad hoc demand.

While the retrieval arm 104 has been described as being movable along the z-axis to move into the container 102, it shall be further appreciated that the retrieval arm 104 may be movable in an x-y plane (defined by x-y axes) away from the plurality of containers 102 supported on the carrier 106. For example, small amounts of movement of the retrieval arm 104 in the x-y plane above the predetermined position 210 may facilitate moving the retrieval arm 104 to a specific portion of the container 102 for the next retrieval attempt.

The retrieval arm 104 may include any one or more of various different features of retrieval arms described, for example, in U.S. Pat. No. 9,731,853, issued on Aug. 15, 2017, and entitled “DISPENSABLE UNIT RETRIEVAL MECHANISM,” the entire contents of which are hereby incorporated herein by reference. As an example, the retrieval arm 104 may include a tube 134 and a nib 136. The tube 134 may be hollow, and the nib 136 may be supported along an end portion of the tube 134. In such implementations, the dispensing device 100 may further include a vacuum device 138 in fluid communication with the nib 136 via the tube 134, with the fluid communication between the vacuum device 138 and the nib 136 controllable to control suction force at the nib 136. That is, with the vacuum device 138 in fluid communication with the nib 136, suction force at the nib 136 may be used to draw pills from the container 102 as the retrieval arm 104 is moved, along the z-axis, into proximity of the pills in the container 102 at the predetermined position 210. In some instances, the suction at the nib 136 may inadvertently draw a fraction of a pill or more than one pill from the container 102 at the predetermined position 210. This may be detected, for example, based on a change in weight of the container 102 in mechanical communication with a weight sensor, before and after the retrieval attempt. In such cases, fluid communication between the vacuum device 138 and the nib 136 may be interrupted (e.g., by turning off the vacuum device 138 and/or by closing one or more valves) to allow the fraction of a pill or the multiple pills to fall from the nib 136 and back into the container 102. Similarly, in instances in which the weight sensor detects a change in weight corresponding to retrieval of a single pill carried on the nib 136 of the retrieval arm, the vacuum device 138 may remain in fluid communication with the nib 136 until the retrieval arm is over the dispensing zone 133, where the fluid communication may then be interrupted to allow the single pill to fall into the dispensing zone 133 from the nib 136. Further, or instead, the vacuum device 138 may be reversible, as may be useful for blowing air out of the nib 136 to facilitate clearing debris from the nib 136. The weight sensor associated with retrieval of pills from the container 102 may include any one or more of various, different features of weight sensors described in in U.S. Patent Application Publication No. US20220202652, published on Jun. 30, 2022, and entitled “SENSING RETRIEVAL OF PILLS,” the entire contents of which are hereby incorporated herein by reference.

Further, or instead, following each successful retrieval attempt of a single pill from the container 102, the retrieval arm 104 may be movable in the x-y plane to move from a position above the predetermined position 210 to a position above the dispensing zone 133, where one or more successfully retrieved pills may be released to the dispensing zone 133 (e.g., to the dispensing cup 109 positioned at the dispensing zone 133).

In certain instances, the dispensing device 100 may include a sensor 135 (e.g. an optical sensor, a weight sensor, or a combination thereof) in electrical communication with the controller 140 and arranged relative to the dispensing zone 133 to detect at least the presence and/or absence of pills at the dispensing zone 133 (e.g., in the dispensing cup 109 at the dispensing zone 133). For example, the sensor 135 may include a strain gauge mechanically coupled to a surface supporting the dispensing cup 109 at the dispensing zone 133.

In general, the controller 140 may be in communicating relationship (e.g., in wired or wireless communication) with one or more of the retrieval arm 104, an actuator of the carrier 106, the vacuum device 138, the sensor 135, and the user interface 107. The controller 140 may, for example, include a processing unit 142, one or more non-transitory computer-readable storage media 144, and a network interface 145. The processing unit 142 may include one or more processors. The one or more non-transitory computer-readable storage media 144 may have stored thereon instructions for causing the processing unit 142 to carry out one or more aspects of any one of the various, different weight-based sensing techniques described herein for sensing retrieval of pills from the plurality of containers 102. Further, or instead, the one or more non-transitory computer-readable storage media 144 may have stored thereon a predetermined schedule associated with the user such that the processing unit 142 may retrieve consumable media according to the predetermined schedule according to any one or more of the various different techniques described herein. In use, the sensor 135 may provide a first signal and/or a second signal to the controller 140 to carry out any one or more aspects of monitoring a user's dosing regimen according to the techniques described herein. In certain implementations, the network interface 145 may couple the controller 140 (e.g., one or more of the processing unit 142 or the non-transitory computer-readable storage media 144) to a data network to couple the controller 140 in communicating relationship with one or more remote resources over a data network, as described in greater detail below with respect to carrying out various aspects of monitoring dosing regimens according to certain techniques described herein.

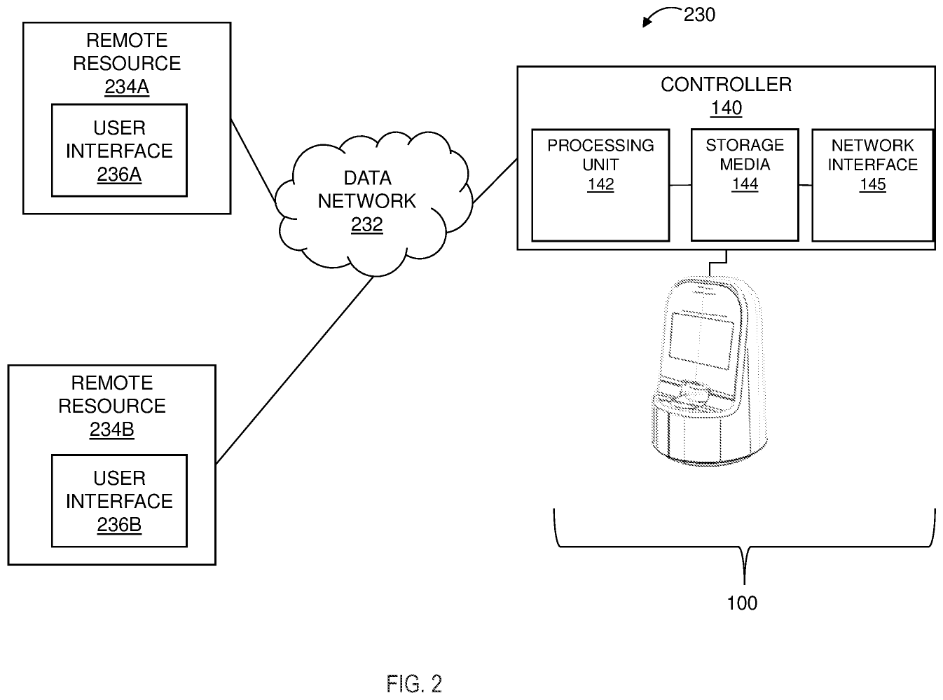

Referring now to FIG. 2, a system 230 may include the dispensing device 100, a data network 232, a remote resource 234A including a user interface 236A and associated with the user, and a remote resource 234B including a user interface 236B associated with a third-party (e.g., a health-care professional, pharmacy, care-giver, authorized family member, manufacturer of the dispensing device 100, etc.). Unless otherwise specified or made clear from the context, it shall be appreciated that the system 230 may carry out any one or more of various, different aspects of monitoring dosing regimens described herein.

In general, the remote resource 234A associated with the user and the remote resource 234B associated with a third-party may include a smart phone, smart watch, a tablet, a computer, or other similar device that includes a user interface providing at least one-way communication and, in certain instances, providing two-way communication between the respective resource and the controller 140 of the dispensing device 100 over the data network 232. In certain implementations, the remote resource 234A associated with the user may be additionally or alternatively in communication with the controller 140 of the dispensing device 100 through one or more types of local communication, such as Wi-Fi or Bluetooth. This may be useful, for example, for a user to communicate with the controller 140 using a remote resource 234A, such as a smart phone, smart watch, or tablet in the home without the need to connect to the data network 232. The user interface 236A of the remote resource 234A and the user interface 236B of the remote resource 234B may each include any one or more of various, different types of interfaces useful for efficient communication of signals, prompts, alerts, and/or other types of communication according to the various different dosing monitoring techniques described herein. Thus, for example, the respective user interfaces may include one or more of a speaker, a graphical user interface, voice-controlled interface, gesture-based interface, a keyboard, a touchscreen, a microphone, a mouse, etc.

Still more generally, a variety of fixed and distributed infrastructures may be usefully employed with system components to provide value-added services and augment operation of the system for an individual user. Thus, portions of the systems and methods described herein may be implemented in the cloud, while other portions use local processing resources for a device, such as a computer, printer, camera, and the like coupled to a local area network shared with the components of the system. In another aspect, other network resources may be usefully combined with cloud-based services and local processing resources to provide various layers of functionality, knowledge sharing, redundancy, and speed. For example, while a cloud-based or other remote-hosted system may manage inventory of content of containers of the dispensing device 100 at appropriate times, this may also be performed with a suitably programmed local computer that can read compliance data, container status, and scheduling information from one or more components of the dispensing device 100 and/or the system 230 and determine when refills of the consumable media might be needed. Similarly various notification systems, monitoring functions, data storage functions, management and administration functions and the like may be distributed or centralized in various manners across available resources according to user preferences, security requirements, oversight required by health care professionals, data integrity requirements and so forth. All such variations are intended to fall within the scope of this disclosure, and not particular function, service, system component, sub-component, or communications interface or endpoint should be presumed to reside with any specific system element unless explicitly stated to the contrary or otherwise clear from the context.

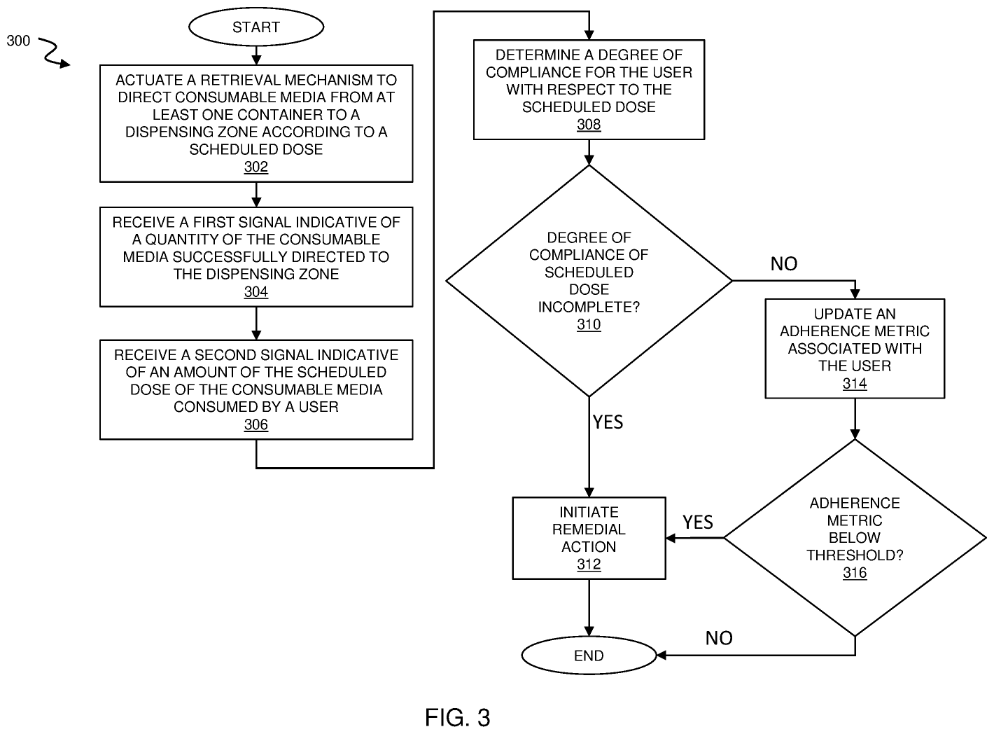

FIG. 3 is a flow chart of an exemplary method 300 for monitoring a dosing regimen of consumables. Unless otherwise specified or made clear from the context, it shall be appreciated that any one or more aspects of the exemplary method 300 may be implemented as computer-readable instructions stored on the non-transitory computer-readable storage media 144 (FIG. 1C) and executable by the processing unit (FIG. 1C) of the controller 140 (FIG. 1C) and/or distributed across any one or more resources of the system 200 (FIG. 2) to monitor a dosing regimen including consumables stored in any one or more of the containers described herein and, further or instead, to monitor consumables that are stored apart from the one or more containers.

As shown in step 302, the exemplary method 300 may include actuating a retrieval mechanism to direct at least a portion of a scheduled dose of consumable media, according to a predetermined schedule, from one or more containers to a dispensing zone of a dispensing device (such as the dispensing device 100 described above with respect to FIGS. 1A-1D and FIG. 2. In general, the dispensing zone may be any portion of the dispensing device in which a portion of the scheduled dose of consumable media is separate from bulk storage of the consumable media. Accordingly, as described in greater detail below, the presence or absence of the portion of the scheduled dose of consumable media in the dispensing zone may be advantageously detected to facilitate monitoring a user's compliance (also referred to as adherence) to a regimen for consuming the consumable media. In certain implementations, a user may retrieve the scheduled dose of consumable media directly from the dispensing zone, as described above with respect to operation of the dispensing device 100 (FIGS. 1 and 2A-2D).

As shown in step 304, the exemplary method 300 may include receiving a first signal indicative of a quantity of the consumable media directed from the one or more containers to the dispensing zone. For example, the first signal may include feedback from one or more sensors disposed along the dispensing zone, such as the sensor 135 (FIG. 1A). In some instances, the one or more sensors may include an optical sensor arranged relative to the dispensing zone to detect at least the presence of consumable media (e.g., an infrared break beam sensor) along the dispensing zone to detect whether a dispensing operation was successful. Additionally, or alternatively, the optical sensor may detect a quantity of consumable media dispensed to the dispensing zone, such as through detection of distribution of light at the dispensing zone. Further, or instead, the one or more sensors may include a weight sensor disposed along the dispensing zone and arranged to detect changes in mass supported by the dispensing zone, with such changes at least indicative of whether a dispensing operation was successfully carried out. Additionally, or alternatively, such changes in mass measured by the weight sensor may be indicative of a quantity of the consumable media dispensed to the dispensing zone, such as may be particularly useful for distinguishing whether a given dispensing event corresponds to distribution of a full dose or a partial dose. Further, in instances in which the dispensing event corresponds to a partial dose, the weight sensor may advantageously provide information that may be used to quantify the fraction of a full dose dispensed.

As shown in step 306, the exemplary method 300 may include receiving a second signal indicative of a quantity of the scheduled dose of the consumable media consumed by a user associated with the scheduled dose. Stated differently, while the first signal may provide an indication of the quantity of consumable media dispensed to a dispensing zone, the first signal alone—without any other information—may have limited probative value in determining whether the user ultimately consumed the consumable media following the dispensing event. Thus, in general, the second signal may include information that supplements the first signal to provide a more robust indication—directly and/or by inference—of whether the user consumed the consumable media following a successful dispensing event. In addition to facilitating improved a baseline (yes/no) determination of whether the user consumed at least a portion a schedule dose of the consumable media, the second signal may be useful for determining details of a user's consumption in certain instances.

In certain instances, the second signal may be indicative of a degree of consumption of the scheduled dose. As used in this context, a “degree of consumption” shall be understood to refer to a fraction of a scheduled dose of the consumable media determined and/or inferred to be ultimately consumed by the user in accordance with a schedule. Further, or instead, the degree of consumption may account for partially dispensed doses determined based on the first signal. For example, the first signal may be indicative of 5 out of 10 pills of a scheduled dose dispensed. Continuing with this example, the second signal may be indicative of 3 out of the 5 dispensed pills as having been consumed. Thus, according to this example, the second signal may be indicative of a degree of consumption of 30 percent. Stated differently, the degree of consumption may reflect the combined effect of two different sources of deviation from a regimen: 1) partial dispensing of a scheduled dose; and 2) partial consumption of a dispensed dose by the user. Thus, as compared to compliance determinations that are “yes/no” in nature, the degree of consumption determined based on the second signal, alone or in combination with the first signal, may facilitate making compliance determinations with more granularity which, in turn, may facilitate providing more informed and/or additional remediation responses to users deviating from dosing regimens of consumable media, as described in greater detail below.

The second signal may further, or instead, be indicative of a time of actual consumption of the at least one portion of the schedule dose of consumable media following dispensing of the consumable media. This may provide temporal resolution with respect to determining a user's compliance to a predetermined dosing regimen. For example, as described in greater detail below, certain remedial actions may be initiated in instances in which timeliness of consumed dosages is detected, via the second signal, as a factor contributing to poor compliance with a dosing regimen.

In certain implementations, the second signal may correspond to removal of the consumable media from the dispensing zone (e.g., following detection of successful dispensing of the consumable media to the dispensing zone). For example, the second signal may include feedback from a sensor detecting removal of a cup (or similar container) from the dispensing zone. That is, in some implementations, it shall be appreciated that the first signal and the second signal may each be measured locally on the dispensing device, as may be useful for cost-effectively monitoring compliance of a particular therapy associated with the consumable media. Continuing with this example, the second signal may be measured by any one or more of the various, different sensors described herein (e.g., an optical sensor, a weight sensor, or a combination thereof).

In some instances, the first signal and the second signal may be measured by different sensors and, in some cases, by different types of sensors. For example, the first signal and the second signal may correspond to signals from sensors with different resolutions. As a more specific example, the first signal may be measured by a sensor with resolution useful for distinguishing a partially dispensed dose from a fully dispensed dose of consumable media, while the second signal may be measured by a sensor with a lower resolution (e.g., a sensor with two-states). In such instances, determining a user's compliance to a therapy may be based on an inference a user removes and subsequently consumes the entire amount of the consumable media that has been detected, based on the first signal, as being dispensed. This inference may facilitate monitoring compliance to a therapy using inexpensive hardware that may be robust over a variety of conditions.

In certain implementations, the same sensor or sensors may be used to measure the first signal and the second signal. For example, detection of a successful dispensing event may distinguish the first signal from the second signal in such cases. More specifically, in instances in which the sensor is a weight sensor arranged to measure weight supported on the dispensing zone, the first signal may correspond to an increase in weight measured by the weight sensor (indicative of dispensing at least a portion of a scheduled dose of consumable media to the dispensing zone), and the second signal may correspond to a subsequent decrease in weight measured by the weight sensor (indicative of removal of the dispensed amount of the consumable media). In this example, it shall be appreciated that the amount of the consumable media removed from the dispensing zone may be inferred as being consumed by the user for the purpose of determining compliance to a therapy. Here, again, it may be appreciated that inaccuracy in compliance tracking using this inference may be acceptable in use cases in which cost-effectiveness is a primary consideration relative to accuracy.

While the second signal may be based on one or more signals received from a sensor or sensors, other sources of the second signal are additionally or alternatively possible. For example, the second signal may include one or more user inputs at a user interface (e.g., a user interface at the dispensing device, a user interface on a remote resource associated with the user, or a combination thereof) in response to one or more prompts on the user interface. For example, following an at least partially complete dispensing event indicated by the first signal, the user interface may prompt the user to confirm that the user has taken the consumable media that has been dispensed. Continuing with this example, the one or more prompts on the user interface may ask the user to input one or more of a type or a number of the consumable media consumed by the user at a scheduled time associated with the scheduled dose of the consumable media. This information may be compared to the scheduled dose to determine the degree of consumption of the consumable media. In some instances, the time of the one or more user inputs received at the user interface may serve as a proxy for the time at which the user took the consumable media. In other instances, the one or more inputs received at the user input may include the time at which the user took the consumable media.

In certain implementations, one or more inputs in the second signal may facilitate modifying the dosing regimen. For example, the one or more prompts at the user interface may ask the user to provide an explanation for lack of compliance. In response to such prompts, the one or more user inputs in the second signal may include an indication that at least a portion of the consumable media is no longer needed. In turn, this information may be communicated to one or more parties, such as the user's doctor, who may contact the user for a consultation. Further or instead, in instances in which the one or more user inputs in the second signal indicate that at least a portion of the consumable media is no longer needed, the dose or partial dose may be tracked as “taken.” That is, the user's response indicating that the scheduled dose of the consumable media is no longer needed may be tracked such that it does not adversely impact the user's degree of compliance related to the scheduled dose and/or the user's adherence metric with respect to the consumable media.

While user inputs may facilitate indirect modification of the dosing regimen in some instances, it shall be appreciated that one or more inputs in the second signal may additionally, or alternatively, may relate directly to user autonomy in managing the dosing regimen. For example, the one or more user inputs in the second signal may include an instruction to exclude at least one of the one or more containers from subsequent attempts to direct the consumable media to the dispensing zone. The instruction may identify the container by position and/or by contents of the container. Thus, through the one or more inputs in the second signal the user may deliberately opt out of compliance with a dosing regimen. In certain instances, such deliberate action on the part of the user may be treated differently from a decrease in compliance determined over time. As an example, in instances in which the one or more inputs to the second signal include an instruction to exclude at least one of the one or more containers from subsequent attempts at dispensing consumable media, compliance tracking may be stopped until the user authorizes retrieval again.

In certain instances, the instruction to exclude at least one of the one or more containers from subsequent attempts may facilitate faster retrieval. For example, in some use-cases, the same type of consumable media may be present in more than one container. In such use-cases, the instruction to exclude one of the containers may result in retrieval from the container that is fuller and, thus, facilitates faster retrieval.

As shown in step 308, the exemplary method 300 may include determining a degree of compliance for the user with respect to the scheduled dose. In general, determining the degree of compliance with respect to the scheduled dose may be based on confirmation and/or determination that a quantity of the consumable media has been successfully dispensed such that the quantity of consumable media is available to the user in the dispensing zone (e.g., based on the first signal), the quantity of a scheduled dose of the consumable media consumed by the user (e.g., based on the second signal), or a combination thereof. In some instances, the quantity of the scheduled dose of the consumable media consumed by the user may be determined to be greater than the quantity of the consumable media dispensed to the dispensing zone in certain instances, such as may be the case in which the dispensed quantity of the consumable media is less than the scheduled dose (e.g., because a container of the dispensing device is low or has become empty) and the user consumed the remainder of the scheduled dose from a storage location separate from the dispensing device (e.g., when a dispensing operation of the dispensing device fails or partially fails for any reason other than the container being empty, the user may be prompted to manually retrieve the consumable media from the container in the dispensing device. More generally, as may be appreciated from the foregoing example, determining the degree of compliance based on a combination of the first signal indicative of the quantity of the consumable media directed to the dispensing zone from one or more containers and the second signal indicative of a quantity of the scheduled dose of the consumable media consumed by the user may be more robust across a wider range of use cases, as compared to using either signal individually. Among other things, such robustness may reduce the likelihood of repeatedly initiating unnecessary remedial actions that tend to lead to user frustration and decreased reliance on automated management of dosing regimens.

In certain implementations, the degree of compliance with respect to a scheduled dose may include a “yes/no” determination corresponding to the categories of compliance and non-compliance. Compliance determinations according to these two categories may facilitate using information acquired locally at the dispensing device as the basis for the first signal and the second signal. That is, determining compliance and non-compliance may facilitate the use of hardware configurations, such as those described above, that may be cost-effectively implemented and further, or instead, may require little or no maintenance.

For some users, however, the categories of compliance and non-compliance with respect to a scheduled dose may provide an incomplete assessment of the user's adherence to a dosing regimen. Given that many users may be neither entirely compliant nor entirely non-compliant, the degree of compliance may additionally, or alternatively, include one or more degrees of partial compliance with respect to the scheduled dose. Importantly, the one or more degrees of partial compliance may distinguish incomplete compliance from inadequate compliance, as may be useful in managing therapeutic treatment that may remain effective at certain levels below complete compliance. Such insight may reduce false positives in remedial action—that is, incidences of premature or inappropriate initiation of remedial action. In turn, such reduction in remedial action may improve a user's experience (e.g., by reducing a likelihood of user frustration) with automated management of a dosing regimen while maintaining therapeutic benefits of the dosing regimen.

As shown in step 310, the exemplary method 300 may include determining whether the degree of compliance with a scheduled dose is incomplete and, optionally, taking action at least on that basis. In general, determining whether the degree of compliance is incomplete may be based on comparing the degree of compliance to a predetermined compliance threshold for the dosing regimen of the consumable media. For example, in instances in which the degree of compliance is determined only according to the categories of compliance and non-compliance, the predetermined compliance threshold may be 100 percent such that anything less than complete compliance is categorized as non-compliance. In other instances, namely those in which the degree of compliance is indicative of partial compliance, the predetermined compliance threshold may be greater than 0 percent and less than 100 percent to reflect that some amount of compliance below 100 percent compliance is acceptable for a particular use case. In general, the predetermined compliance threshold may vary according to factors including, but not limited to, type of consumable media, the dose of the consumable media, one or more physical characteristics of the user, other types of consumable media in the dosing regimen for the user, physician preference, or a combination thereof.

As shown in step 312, the exemplary method 300 may include initiating any one or more of various, different remedial actions when the degree of compliance with the scheduled dose is below the predetermined compliance threshold such that the degree of compliance is determined to be incomplete for the user with respect to the scheduled dose. In general, initiation of the remedial action based on the degree of compliance with the scheduled dose may be directed toward improving the user's treatment according to the dosing regimen. In some cases, such improvement may include increasing the likelihood that the user will take the scheduled dose or take some additional amount of the scheduled dose, such as through reminding the user and/or making consumption of the scheduled dose more convenient for the user. Further, or instead, the remedial action may improve the user's treatment according to the dosing regimen by promoting an efficient framework for discussion between the user and a healthcare provider regarding changes to the dosing regimen deliberately or unintentionally initiated by the user.

In some instances, the remedial action may include communicating the user in one-way or two-way communication via a user interface associated with the user. Here, a user interface associated with the user may include a user interface accessible by the user using log-in credentials, biometric information, or other types of authentications of identity of the user. Further, or instead, the remedial action may include sending one or more prompts to a user interface associated with the user. In certain implementations, the user interface associated with the user may include a user interface on the dispensing device itself. Additionally, or alternatively, the user interface associated with the user may be on a remote device associated with the user (e.g., a smart phone, smart tablet, laptop, personal computer, or other remote device associated with the user and in communication with the dispensing device via a data network).

As an example, the remedial action may include sending one or more alerts to a user interface associated with the user. These alerts may include audio and/or visual indications that the scheduled dose has been dispensed and reminding the user to ingest the consumable media at a scheduled time. Further, or instead, the one or more alerts may change with increasing time that the degree of compliance of the scheduled dose is incomplete. For example, the alert may initially be provided to a user-interface on the dispensing device. If the alert condition persists past a predetermined period, the alert may be promulgated to remote resources including respective user interfaces associated with the user.

The remedial action may include sending one or more prompts to a user interface associated with the user. The one or more prompts may be sent to a user interface on the dispensing device such that the user may interact locally with the dispensing device. Further, or instead, the one or more prompts may be sent to an application on a remote device associated with the user, as may be useful for facilitating interaction with the user when the user is away from the dispensing device. Further, or instead, the remote device may have a familiar input interface (e.g., touch keyboard) familiar to the user and, thus, useful for gathering detailed information from the user in response to the one or more prompts.

One or more user inputs in response to the prompts may form at least a portion of the second signal indicative of a quantity of the scheduled dose of the consumable media consumed by a user associated with the scheduled dose. That is, importantly, by incorporating user input into the second signal and ultimately into the degree of compliance based on the first signal and the second signal, the degree of compliance may be more accurately tracked, as compared to relying only on the first signal indicative of dispensing the scheduled dose to the dispensing zone. That is, with user input incorporated into the second signal, the degree of compliance may be determined with fewer inferences, as compared to determining a degree of compliance based only on information associated with dispensing. For example, the one or more user inputs in the second signal may correspond to one or more of a type or a number of the consumable media consumed (ingested, applied, etc.) by the user at a scheduled time. This may serve as confirmation that the user is in compliance or at least partial compliance with the scheduled dose of the consumable media according to the dosing regimen. Continuing with this example, the one or more user inputs in the second signal may include an indication that the dispensing device is not functioning properly, thus facilitating efficient troubleshooting. Further, or instead, the one or more user inputs in the second signal may include an indication that at least a portion of the consumable media is no longer needed, thus potentially resulting in a change to the dosing schedule and/or prompting a discussion between the user and a healthcare provider regarding the degree of compliance of the scheduled dose being incomplete.

In certain implementations, incorporating user input into the second signal indicative of a quantity of the scheduled dose consumed by the user may facilitate accommodating different use cases. Such use cases may make the dispensing device more useful to a wider population of users. Further, or instead, incorporating the user input into the second signal indicative of quantity of the scheduled dose consumed may reduce the likelihood of individual user frustration with automated dispensing of consumable media according to a dosing regimen. This may, in turn, promote improved user adherence to the dosing regimen and, ultimately, may improve user outcome with respect to the therapy associated with the dosing regimen.

As an example, the scheduled dose of the consumable media may include dose information for one or more consumables stored apart from the one or more containers and separately accessible by the user. This may be useful, for example, for tracking a user's compliance with a dosing regimen for a consumable that must be stored under specific conditions (e.g., refrigerated). Under such conditions, it may be more convenient for the user to retrieve the one or more consumables from containers separately accessible by the user apart from the dispensing device while incorporating the user inputs into the second signal such that the degree of compliance (and/or the adherence metric based on the degree of compliance) may provide a complete assessment of the user's compliance, independent of whether the user obtained all or a portion of a dose from a source separate from the dispensing device.

As another example, incorporating user input into the second signal indicative of a quantity of the scheduled dose consumed by the user may facilitate, for example, assisting the user in adhering to the dosing regimen when the user is typically away from the dispensing device (e.g., when the user is traveling, at work, at school, etc.). that a user must take when the user is typically away from the dispensing device (e.g., at school, at work, etc.). For example, a scheduled dose of the consumable media may be dispensed to the dispensing zone in advance of the time at which the dose is scheduled to be consumed by the user. This may allow the user to pack the scheduled dose for later consumption while the user is away from the dispensing device. Continuing with this example, the first signal indicative of at least partial dispensing of the scheduled dose may provide confirmation that at least a portion of the scheduled dose was successfully dispensed. However, at this stage, the determination of degree of compliance may remain pending until the user input in the second signal is received. Continuing with this example, one or more prompts may be provided to a user interface on a remote resource associated with the user (e.g., reminders sent to a mobile app on a smart phone associated with the user) at a scheduled time for consuming the consumable media. In response to the prompt, the user may provide user input confirming that at least a portion the consumable media. This input may be at least a portion of the second signal indicative of a quantity of the scheduled dose of the consumable media consumed by the user associated with the scheduled dose. Thus, following user confirmation that the pre-dispensed dose has been consumed at the scheduled time, the degree of compliance of the scheduled dose may be determined as compliant or partially compliant, as dictated by the user input. It shall be appreciated that, conversely, if the user does not confirm consumption at the later time (e.g., by not replying to a prompt or affirmatively acknowledging that the dose was not taken), the degree of compliance of the scheduled dose may be determined to be non-compliant and any one or more of the various, different remedial actions described herein may be initiated.

In some implementations, the one or more prompts sent to the user interface may be state-dependent on the dispensing device. For example, a first set of prompts may be sent to the user interface if the first signal indicates that at least one of the one or more containers of the dispensing device is empty and otherwise sending a second set of prompts, different from the first set of prompts, if the first signal indicates that the quantity of the consumable media directed to the dispensing zone is less than the scheduled dose of the consumable media. More specifically, if it is determined that the user's degree of compliance may be below the predetermined compliance threshold for the scheduled dose because there is an insufficient amount of the consumable media in the one or more containers, the first set of prompts may prompt the user to contact the user's doctor or pharmacy to order a refill of the consumable media, thus streamlining the user's experience with respect to maintaining the consumable medial within the dispensing device. Continuing with this example, if the user declines to move forward with ordering a refill, the second set of prompts may be sent to the user interface to facilitate gathering more information about the user's experience and, to the extent appropriate, initiating one or more additional remedial actions.

More generally, as may be appreciated from the foregoing example, the prompts sent to the user interface associated with the user may be useful for determine an underlying reason for the user's incomplete compliance with respect to the scheduled dose. As an example, the prompts may elicit information from the user indicating that the user has decided not to continue taking the consumable media or has decided to cut back on the dose of the consumable media. This information may be relayed to a remote resource associated with a healthcare professional associated with the user (e.g., the user's doctor, physician's assistant, nurse practitioner, pharmacist, etc.) to make the healthcare professional aware of the change and provide the healthcare professional with an opportunity to confer with the user as necessary. As compared to intervention by a healthcare professional only after an adverse event or at a next scheduled appointment, the user's responses to the prompts may facilitate earlier interventions in managing the user's dosing regimen and, ultimately, may improve outcomes of user treatment according to the dosing regimen.

Additionally, or alternatively, the prompts sent to the user interface associated with the user may be initiate one or more remedial actions associated with troubleshooting the dispensing device. For example, in instance in which there is a sufficient amount of the consumable media in a container of the dispensing device and the first signal is indicative of failed or incomplete dispensing of the consumable media to the dispensing zone, the prompts may instruct the user to open the dispensing device to retrieve the consumable media from the container and ask the user to confirm taking the full dose of the consumable media.

Further or instead, as shown in step 314, the exemplary method 300 may include updating an adherence metric (e.g., a percentage of compliance with respect to a dosing regimen of consumables) according to the degree of compliance. It shall be appreciated that while the degree of compliance for the scheduled dose relates to a given dose in a scheduled regimen that is being managed by a dispensing device, the adherence metric is an indication of a user's overall adherence to the scheduled regimen over some therapeutically relevant period (e.g., day, week, month, etc.) for the treatment provided by the consumable media. Thus, in cases in which each dose of the consumable media is critical for the user's treatment, the degree of compliance for the schedule dose and the adherence metric may be the same. In many cases, however, the degree of compliance and the adherence metric may correspond to different periods of time, with the degree of compliance being a snapshot of user behavior associated with a single dose and the adherence metric being reflective of one or more trends in user behavior. Each metric, therefore, may be useful individually or in combination with one another initiating appropriate remedial action to maintain or improve delivery of the dosing regimen to the user.

In certain instances, the adherence metric may be updated following one or more remedial actions. That is, through one or more remedial actions, the user may be brought into compliance with respect to the scheduled dose. Since the degree of compliance is an input to the adherence metric, the adherence metric may be updated (e.g., increased) accordingly to reflect that the one or more remedial actions were effective, and the user has taken the scheduled dose.

The adherence metric may be updated when the degree of compliance for a scheduled dose is complete and/or at least above a certain threshold (e.g., a predetermined threshold), in the case of partial compliance. The adherence metric may be specific to one type of consumable media dispensed to the user and, in cases in which a user is dispensed different types of consumable media as part of a dosing regimen, the user may receive a respective adherence metric for each type of consumable media. Further, or instead, adherence metrics for different types of consumable media may be combined to provide the user or another party with an indication of overall compliance.

In certain implementations, the updated adherence metric may be sent to one or more stakeholders. For example, the updated adherence metric may be sent to the user interface over a data network and/or through wired communication, as the case may be with respect to communication between the user interface and one or more processors, to provide the user with an indication of progress and/or regression with respect to adherence to a dosing regimen of consumables. Further, or instead, the updated adherence metric may be sent to one or more remote resources, such as remote resources associated with one or more of the user's doctors, caregivers, and/or family members, as may be useful for facilitating intervention by one or more of these stakeholders if the updated adherence metric indicates one or more missed doses, falls below a therapeutic threshold, etc.

In certain implementations, the update to the adherence metric may occur substantially in real-time, allowing for finite processing and communication speed between a user interface and one or more processors carrying out the exemplary method 300. Among other things, such real-time updating of the adherence metric may be useful for modifying the schedule prior to the user's next scheduled dose. Further, or instead, real-time updating of the adherence metric may be useful for reducing the likelihood of medical complications that may result from compliance falling below a threshold. That is, with the adherence metric updated substantially in-real time, the user may have an opportunity to take corrective action before the onset of any medical complications.

As shown in step 316, the exemplary method 300 may include determining whether the adherence metric is below an adherence threshold and, optionally, initiating one or more of the remedial actions based on the adherence metric. Unless otherwise specified or made clear from the context, it shall be appreciated that the one or more remedial actions initiated based on the adherence metric being below an adherence threshold include any one or more of the adherence metrics that may be initiated with respect to the degree of compliance of the scheduled dose being determined to be incomplete. For example, if the adherence metric falls below the adherence threshold, a remedial action may include sending one or more prompts to a user interface associated with the user. These prompts may be the same as the prompts associated with the remedial action associated with an incomplete degree of compliance and, thus, may be indistinguishable to the user. However, in certain instances, the prompts associated with the remedial action in response to the adherence metric being below an adherence threshold may be different (e.g., asking for more extensive information from the user) from the prompts related to an incomplete degree of compliance. Further, or instead, a remedial action in response to the adherence metric falling below an adherence threshold may include contacting a healthcare provider or other stakeholder in instances in which an incomplete degree of compliance may not result in such remedial action by itself.

The above systems, devices, methods, processes, and the like may be realized in hardware, software, or any combination of these suitable for a particular application. The hardware may include a general-purpose computer and/or dedicated computing device. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software. In another aspect, the methods may be embodied in systems that perform the steps thereof and may be distributed across devices in a number of ways. At the same time, processing may be distributed across devices such as the various systems described above, or all of the functionalities may be integrated into a dedicated, standalone device or other hardware. In another aspect, means for performing the steps associated with the processes described above may include any of the hardware and/or software described above. All such permutations and combinations are intended to fall within the scope of the present disclosure.

Embodiments disclosed herein may include computer program products comprising computer-executable code or computer-usable code that, when executing on one or more computing devices, performs any and/or all of the steps thereof. The code may be stored in a non-transitory fashion in a computer memory, which may be a memory from which the program executes (such as random-access memory associated with a processor), or a storage device such as a disk drive, flash memory or any other optical, electromagnetic, magnetic, infrared or other device or combination of devices. In another aspect, any of the systems and methods described above may be embodied in any suitable transmission or propagation medium carrying computer-executable code and/or any inputs or outputs from same.

It will be appreciated that the devices, systems, and methods described above are set forth by way of example and not of limitation. Absent an explicit indication to the contrary, the disclosed steps may be modified, supplemented, omitted, and/or re-ordered without departing from the scope of this disclosure. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context.

The method steps of the implementations described herein are intended to include any suitable method of causing such method steps to be performed, consistent with the patentability of the following claims, unless a different meaning is expressly provided or otherwise clear from the context. So, for example performing the step of X includes any suitable method for causing another party such as a remote user, a remote processing resource (e.g., a server or cloud computer) or a machine to perform the step of X. Similarly, performing steps X, Y and Z may include any method of directing or controlling any combination of such other individuals or resources to perform steps X, Y and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It should further be appreciated that the methods above are provided by way of example. Absent an explicit indication to the contrary, the disclosed steps may be modified, supplemented, omitted, and/or re-ordered without departing from the scope of this disclosure.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims, which are to be interpreted in the broadest sense allowable by law.

What is claimed is:

1. A method of monitoring a dosing regimen of consumables, the method comprising:
actuating a retrieval mechanism to direct at least a portion of a scheduled dose of consumable media, according to a predetermined schedule, from one or more containers to a dispensing zone;
receiving a first signal indicative of a quantity of the consumable media directed from the one or more containers to the dispensing zone;
receiving a second signal indicative of a quantity of the scheduled dose of the consumable media consumed by a user associated with the scheduled dose; and
based on the quantity of the consumable media directed to the dispensing zone and the quantity of the consumable media consumed by the user, determining a degree of compliance for the user with respect to the scheduled dose.

2. The method of claim 1, wherein the degree of compliance includes non-compliance, compliance, and one or more degrees of partial compliance with respect to the scheduled dose.

3. The method of claim 1, wherein the first signal includes feedback from an optical sensor, a weight sensor, or a combination thereof.

4. The method of claim 1, wherein the second signal corresponds to removal of the consumable media from the dispensing zone.

5. The method of claim 1, wherein the second signal includes feedback from an optical sensor, a weight sensor, or a combination thereof.

6. The method of claim 4, wherein the second signal includes feedback from a sensor detecting removal of a cup from the dispensing zone.

7. The method of claim 1, wherein the scheduled dose of consumable media includes dose information for one or more consumables stored apart from the one or more containers and separately accessible by the user.

8. The method of claim 1, further comprising initiating a remedial action when the degree of compliance is incomplete for the user with respect to the scheduled dose.

9. The method of claim 8, wherein the remedial action includes sending one or more alerts to a user interface associated with the user.

10. The method of claim 8, wherein the remedial action includes sending one or more prompts to a user interface associated with the user.

11. The method of claim 10, wherein the remedial action includes sending the one or more prompts to an application on a remote device associated with the user.

12. The method of claim 10, wherein sending the one or more prompts to the user interface includes sending a first set of prompts if the first signal indicates that at least one of the one or more containers is empty and otherwise sending a second set of prompts, different from the first set of prompts, if the first signal indicates that the quantity of the consumable media directed to the dispensing zone is less than the scheduled dose of the consumable media.

13. The method of claim 10, wherein the second signal includes one or more user inputs at the user interface in response to the one or more prompts.

14. The method of claim 13, wherein the one or more user inputs in the second signal correspond to one or more of a type or a number of the consumable media consumed by the user at a scheduled time associated with the scheduled dose of the consumable media.

15. The method of claim 13, wherein the one or more user inputs in the second signal include an indication that at least a portion of the consumable media is no longer needed.

16. The method of claim 13, wherein the one or more user inputs in the second signal include an instruction to exclude at least one of the one or more containers from subsequent attempts to direct the consumable media to the dispensing zone.

17. The method of claim 8, further comprising updating an adherence metric according to the degree of compliance following the remedial action, the adherence metric associated with the dosing regimen of consumables associated with the user.

18. A computer program product encoded on one or more non-transitory computer storage media, the computer program product comprising instructions that, when executed by one or more computing devices, cause the one or more computing devices to perform operations comprising:
receiving a first signal indicative of a quantity of consumable media directed from one or more containers to a dispensing zone;
receiving a second signal indicative of a quantity of the consumable media consumed by a user associated with a scheduled dose of the consumable media; and
based on the quantity of the consumable media directed from the one or more containers to the dispensing zone and the quantity of the consumable media consumed by the user, determining a degree of compliance for the user with respect to the scheduled dose.

19. A dispensing device comprising:
a plurality of containers;
a retrieval arm;
a positioner coupled to the retrieval arm and actuatable to move the retrieval arm in one or more of the plurality of containers; and
a controller in communicating relationship with the positioner, the controller configured to
actuate the positioner to direct at least one portion of a scheduled dose of consumable media, according to a predetermined schedule, from one or more of the plurality of containers to a dispensing zone,
receive a first signal indicative of a quantity measured by a sensor,
receive a second signal indicative of an input from a user interface, and
based on the first signal and the second signal, determine a degree of compliance for a user associated with the scheduled dose.

20. The dispensing device of claim 19, wherein the degree of compliance includes non-compliance, compliance, and one or more degrees of partial compliance with respect to the scheduled dose.
